(19) United States
(12) Patent Application Publication
(10) Pub. No.: US 2002/0013280 A1
(43) Pub. Date: Jan. 31, 2002

(54) Pharmaceutical composition for preventing and treating sexual dysfunction and vasculargenic disease comprising icariin

(76) Inventor: Zhongcheng Xin, Beijing (CN)

Correspondence Address: M. Wayne Western, THORPE NORTH & WESTERN, L.L.P., P.O. Box 1219, Sandy, UT, 84091-1219, US

(21) Appl. No.: 09/859,910

(22) Filed: May 17, 2001

Related U.S. Application Data
(62) Division of application No. 09/596,109, filed on Jun. 16, 2000, now abandoned.

(51) Int. Cl. A61K 31/7048

(52) U.S. Cl. 514/27

(57) ABSTRACT

A pharmaceutical composition containing icariin as an active component for preventing and treating sexual dysfunction and vasculargenic disease is disclosed as is the use of icariin as an active component for the manufacture of such pharmaceutical compositions. Also disclosed is a method for the prevention or treatment of sexual dysfunction and vasculargenic disease which comprising administrating an effective amount of icariin or a pharmaceutical composition containing icariin to a patient who stands in need of such treatment.

### FIELD OF THE PRESENT INVENTION

The present invention relates to pharmaceutical composition for preventing and treating a sexual dysfunction and a vasculargenic disease comprising icariin as an active component, a use of icariin for the manufacture of the pharmaceutical for the prevention or treatment of a sexual dysfunction and a vasculargenic disease and a method of the prevention or treatment of a sexual dysfunction and a vasculargenic disease which comprising administrating a prevention or treatment amount of icariin or a pharmaceutical composition containing icariin to a patient who needs.

### BACKGROUND OF INVENTION

Male sexual function includes sexual desire, penile erection, ejaculation and orgasm. Sexual dysfunction, impotence, can result from a trouble in any one of these systems. These include loss of sexual desire, inability to maintain an erection, premature ejaculation, and lack of emission and inability to achieve orgasm. Frequently, more than one of these problems presents themselves simultaneously.

According to statistics, it has been reported that although there are some differences between age group, approximately 20-60% of adult men suffer from sexual dysfunction, which a higher prevalence according to an increase in age. Common cause of such sexual dysfunction before a mere ten years has been considered a psychogenic disease. As a modern medical science have been developed, it has been found from more than approximately 50% of patients having sexual dysfunction that impotence have resulted from organic cause such as vascular, neurological and neuroendocrine diseases including diabetics, hypertension, and taking medicine, ect.

As the physiology of penis erection has recently been elucidated, substances that induce a relaxation effect on corpus cavernosum of human penis have been used in diagnosing and treating erectile dysfunction. For example, papaverine, phentolamine and prostaglandin E1 have been clinically and widely used for intracavernosal injection therapy. Papaverine is an opium alkaloid and it relaxes smooth muscle in the cavernosum and blood vessels. Phentolamine is an adrenergic inhibitor, while physostigmine and neostigmine are cholonergic agents. However, these agents are restrictedly used because of frequency of undesirable effects including a pain, a prolonged erection. A cavernosum fibrosis and a systematic side effect during the treatment of patients. Further more, trazodone, yohimbin and the like have been known as oral agents, but therapeutic mechanism of these drugs is unclear, and they have a low efficacy and high side effect.

Recently, a phosphodiesterase V inhibitor, sidenafil, has taken an increasing interest in the treatment of sexual dysfunction, but such therapy merely induces a transient erection by using a chemical agent, are too expensive and may cause undesirable systematic effects including flashing, headache hypertension and cardiac problem. In particular, a number of deaths are reported due to the aggravation of cardiopathy. Therefore, uses of this agent are also restricted clinically.

In these situations, it has been requested to develop a safe and effective therapeutic agent for sexual dysfunction in order to enhance an instinct erectile function of human body. Recent studies have been taken a growing interest in a therapeutic agent for sexual dysfunction having high relaxation effects on corpus cavernosum by increasing the production and activity of nitric oxide(NO) and cGMP of corpus cavernosum smooth muscle.

Galenic complex therapies for treating sexual dysfunction by using a tonic have been introduced in a traditional medicine, but actual medical experiment on their therapeutic mechanism have not yet been made. Each single component of the galenic drug has been separated and formulated into pharmaceutical composition, but studies on physiological mechanism and clinical effects have not actively made.

### DISCLOSURE OF INVENTION

Thus, the present inventor has extensively investigated the available literatures and has continuously conducted studies and experiment related to overcome the problem of sexual dysfunction. As a result, it has been found that if an active component, icariin, isolated from a certain Epimedii herba are formulated into pharmaceutical composition. It was a strong relaxation effect on the smooth muscle of corpus carvernosum of penis and a relaxation offect on the smooth muscle of large artery. The pharmacological activity of icariin is relative to the activity enhancement of nitric oxide (NO)-cGMP pathway in smooth cell and thus icariin can be effectively and safely used as a medical agent for preventing and treating a sexual dysfunction and a vasculargenic disease through several animal experiments and clinical experiments. The present invention is bases on such discover.

### DETAILED DESCRIPTION OF INVENTION

The present invention relates to a pharmaceutical composition for preventing and treating a sexual dysfunction and a vasculargenic disease comprising a therapeutically effective amount of icariin as an active component.

The present invention also relates to a use of icariin for the manufacture of pharmaceutical for the prevention or treatment of a sexual dysfunction and a vasculargenic disease.

The present invention further relates to a method of the prevention or treatment of a sexual dysfunction and a vasculargenic disease which comprising administrating a prevention or treatment amount of icariin or a pharmaceutical composition containing icariin to a patient who needs.

Icariin (C₃₃H₄₀O₁₅, molecular weight: 676.67) is a flavonoid isolated from Epimedii herba which has been traditionally used as a nutrient tonic drug. Icariin is believed to be main active component of Epimedii herba. Small amount of other components such as magnoflorine, querectin, epimedin A, B and C were also recently detected by HPLC analysis from Epimedii herba. Icariin has been used mainly as a standard chemical test agent. A few pharmaceutical studies of icariin on enhancing immunomodulatory effect, antihepatotoxic activity, anti-fatigue and prevention of aging were reported recently, but no detailed therapeutic mechanism and effects of icariin for enhancing sexual function has been reported yet.

The present inventor has found that icariin isolated from Epimedii herba has a relaxation effect on corpus cavernosum smooth muscle to improve a sexual function as well as a relaxation effect on vascular smooth muscle and artery blood vessel. Further, icariin has no side effect in the treatment of sexual dysfunction and can be effectively and safely applied to patients suffer from erectile dysfunction combined with diabetic and hypertension.

Penile erection is a hemodynamic process involving relaxation of smooth muscle of corpus cavernosum and its associated arterioles. This relaxation process results in an increased flow of blood into the trabecular spaces of cavernosa. The process can be summarized as follows: The penile blood flow increases as arterial and arteriolar are expended due to various sexual stimuli under control of neurologic and neuroendocrine and vascular system. Relaxing corpus cavernosal smooth muscle to thereby facilitate to increase penile blood flow expands the sinusoid. As intracavernosal pressure increase, the subtunical venular plexus between tunica albuginea and sinusoid is compressed for to strangulation of venous outflow, which induces penile erection sufficient to intravaginal penetration and maintains erection until it reach the orgasm and ejaculation.

The recent studies about the elusive agent of penile erection have led to nitric oxide(NO), a gaseous messenger agent. In this connection, it has been found through animal experiments that nitric oxide(NO) plays an iportent role on penile erection. During sexual stimulation, NO is synthesized in the nerve terminals of parasympathetic and non-adrenergic, non-cholinergic(NANC) neurons in the penis and also by the endothelial cell lining blood vessels and lucunar spaces of corpus cavernosum. NO activates guanylate cyclase resulting in an increased conversion of guanosine triphosphate(GPT) to cyclic guanosine monophosphate(cGMP). cGMP provides the signal, which leads to relaxation of smooth muscle of the corpus cavernosum and penile arteries. A balance between the rate of synthesis regulates the level of cGMP by guanylate cyclase and the rate of hydrolytic breakdown to guanosine 5′-monophosphate(GMP) by cyclic neuclase phosphodiesterase(PDE) isozymes. Therefore, agents for inhibiting cGMP hydrolysis may increase the cGMP signal and they may enhance relaxation of smooth muscle in the corpus cavernosum and thereby facilitate penile erectile responses.

The pharmaceutical composition according to the invention may be formulated from a conventional method by combining icariin as an active component with a pharmaceutically acceptable carrier or an excipient such as starch, lactose, organic solvent and the like, and it can be administered in amounts of from 10-500 mg per dose. Beyond this amount range, the formulation and efficacy may be changed. The dose unit includes tablet, pill, capsules, granular, dispersions, ointments, solution, patch, cream, chewing gum, spray or injections, but may be any other convenient dosage form. The preferable route of administration includes oral, muscle and hypodermic injection, subcutaneous, rectum, urethra, vaginal and vein, but any other convenient mode of administration may be used. The icariin compound is advantageously administered 1 to 4 times a day.

The following example and experiments will more specifically illustrate the present invention. However, it should be understood that the present invention id not limited by these examples in any manner.

### EXAMPLE 1

### Extraction of icariin

Methanol extract of icariin

Dried arial parts of Epimedii herba(500 kg) was extracted 3 times with 10 L of MeOH per 3 days. The solvent was concentrated under reduced pressure to yielded 1.5 L of methanol extract. The MeOH extract was then suspended in water and extracted four times with an equivalent amount of hexan. The water layer was concentrated to be ⅓. To this water layer, 5% citric acid was added, suspended(pH=10) and extracted with an equivalent amount of CHCl₃. The extraction procedures were repeated three times. The water layer of ammonia alkalinity was extracted three times with an equivalent amount of butanol to give 75 g of icariin rich fraction(icariin content=8%).

Ethanol extract of icariin

Dried arial part of Epimedii herba(500 g) was extracted four times with the 20 times amount of ethanol per hour at 80° C., filtered to recover ethanol, suspended with the four times amount of distilled water in ethanol extract, stood for three hours at −30° C., and filtered. The filtrate was further extracted four times with buthanol. Filtered, concentrated and absorbed to polyamide column. These were repeatedly washed with 30% ethanol. The washing solution was mixed and concentrated to which 70% ethanol were added in the 20 times amount. The mixture stood for eight hours and filtered to give a pale yellow extract. The resulting extract was recrystallized from a dry ethanol to obtain 65 g of total flavonoid(Icariin content=52%)

Isolation of icariin

The icariin fraction (icariin rich fraction or total flavonoid) obtained in the above extraction step (a) was dissolved in methanol, absorbed and dried in about 100 g of silica gel(product from Merk Co.) Then fraction was subjected to a silica gel column chromatography to isolate icariin. The resulting substance was then filtered through silica gel column(8×60 cm) and extracted in the order of CHCl₃/MeOH=1, 5:1, 4:1, 3:1, 2:1. The resulting solution was concentrated and added to a mixed solution of methanol/water to give a yellow crystal. This crystal was recrystallized from ethanol. It was developed at UV ray light, showed positive on Pauly reagent and was a yellow crystal having TCL Rf value of 0.6. This proves that it is a pure icariin.

Experiment 1: Safety test

To investigate an acute toxicity of icariin, icariin was administered to SD rat. One group consisted 5 male and female rats, respectively. Each group includes the maximum dosage group 3.0 g.kg, the medium dosage group 2.0 g/kg, and the sub-medium dosage group 1.0 g/kg, The minimum dosage group 0.5 g/kg and the comparative group (physiological saline solution). Each of the teat drugs was once administered subcutaneously. After 7 days, it was shown that LD₅₀ is male 1.88 g/kg and female 1.96 g/kg. These results confirm that icariin has low toxicity.

Experiment 2: Effects on penis cavernosal smooth muscle relaxation and artery motion of icariin

Corpus cavernosum of rabbit, animal having a function similar to human penis, was separated. The relaxation effects (in vitro of icariin rich fraction and pure icariin single component on corpus cavernosal smooth muscle were evaluated. The effects on artery motion of icariin by means of rabbit's main artery were also evaluated.

(a) Preparation of animal tissue

Sixty adult male New Zealand white rabbits(weight2.5-3.0 kg) were used as a test animal. Rabbits were anesthetized with sodium pentobarbital(50 mg/kg). Rabbits penises were removed and corpus cavernosum tissues were carefully dissected free from surrounding tunica albuginea and abdominal aortas were separated from abdominal area.

(b) Preparation of tissue in organ chambers

Corpus cavernosum and abdominal aorta were separated in container with Tyrode(wherein a mixed gas of 95% of O₂ and 5% of CO₂) under microscope for dissection. Strips (size: 2×2×6 mm) were made and abdominal aorta round strips (width: 4 mm) were also used. Each end of corpus cavernosal muscle strips were fixed in 10 ml organ chambers containing Tyrode solution and continuously aerated with mixed gas(95% O₂ and 5% CO₂). The upper end of each strip was suspended from an isometric force displacement transducer(TSD 105, Biopac systems, Santa Barbara, Calif., USA) and the abdominal aorta rings were connected to organ chamber using two stainless steel hooks in the same manner. The water keeping warm between dual layers of organ chamber was continuously circulated to maintain the temperature of solution in bath to 37° C.

The tension of corpus cavernosual muscle strips and abdominal aorta rings were measured and recorded by polygraph (MP100WS: Biopac systems, Santa Barbara, Calif., USA) on a computer.

The initial resting tension of corpus cavernosal and abdominal aorta rings were set at 2 gm. And the tissues were allowed to equilibrate for 1.5-2 hours. Sub-maximal contractile responses to phenylephrine(PHE; 5×10⁻⁶M) were observed. The tissues were then allowed to return to baseline tension by repeatedly washing for 30 minutes with Tyrode solution.

The optional isometric contraction was determined as values of amplitude of the contractions were within 10% of previous 3 consecutive contractions. Each strip was used for up to four separate rounds of testing, washed three times with Tyrode solution, and allowed to equilibrate for 30 minutes between rounds.

(c) Organ chamber experiment

To evaluate the effect of icariin and icariin rich fraction in basal state of corpus cavernosum and artery strips, the isometric tension changes were recorded with icariin from 10⁻¹⁰ and icariin rich fraction from 01 mg/ml on commutative increments after treating muscle strips in basal equilibrium state. To evaluate the relaxation effect of icariin and icariin rich fraction on corpus cavermosum and artery strips when the muscle strips stabilized after precontracted by PHE(5×10⁻⁶M), icariin and icariin rich fraction were administered in increasing concentrations(icariin from 10⁻¹⁰M and icariin rich fraction from 01 mg/ml) and the isometric tension changes were recorded.

To investigate the mechanisms of the relaxation effect of icariin on corpus cavrnosum, the responses of icariin were studied in de-endothialized muscle strips of corpus cavrnosum and precontracted by nitric oxide scavenger N^{ω}-nitric-L-arginine(L-NNA; 10⁻⁵), guanylate cyclase antagonist methylene blue(10⁻⁴ M) and pyrogallol(10⁻⁴ M), soluble guanylate cyclase inhibitor 1H-[1,2,4]oxadiazolo[4,3,-a]quinoxalin-1-one(ODQ; 10⁻⁵ M). The relaxation effect or icariin on corpus cavernosal strips was studied by pretreatment with prostagladine inhibitor indomethacine (10⁻⁵ M) and also muscarine receptor antagonist atropine(10⁻⁴M).

Disruption of the endothelium was achieved by rubbing the corpus cavernosal strips with a dry paper towel between thumb and index finger for 10 seconds. Removal of the endothelium was confirmed by the absence of the relaxation response of corpus cavrnosal strips to acetycholine(10⁻⁵M). L-NNA, Methyline blue, ODQ, indomethacine and atropine were added to the corpus cavernosal strips precontracted by PHE and incubated for 20 minutes prior to the addition of icariin. Pyrogallol(10⁻⁴ M) was added to the corpus cavernosal strips precontracted by PHE and incubated for 5 minutes prior to the addition of icariin.

(d) Test substances and Solvents

Phenylephrine hydrochloride, acetylcholine chloride, atropine sulfate, pyrogallol, N^{ω}-nitric-L-arginine, indomethacin, 1H-[1,2,4]oxadiazolo[4,3,-a]quinoxalin-1-one was from Sigma Chemical Co.(St. Louis, Mo., USA) products and methylene blue was Mallinckrodt Chemical Co.(St. Louis, Mo., USA) product. Tyrode solution Na⁺(153.6), K⁺(5.3), Ca⁺⁻(3.0), Mg⁺⁺(1.2), Cl⁻(157.2), H₂PO₄²⁻-(0.6), SO₄-(1.2), HCO₃(7.1), glucose( 11.4).

(e) Statistical Analysis

Data were analyzed with computer(Acqkowledge 3.2 program, Biopac systems, USA) and all relaxation responses were expressed as a percentage of maximal relaxation, which was distance between the PHE-induced maximal contraction point and the largest downward deflection in the tracing for any given experiment. The statistical analysis was performed using concentration were analyzed using logistic regression analysis. P values less than 0.05 was taken to represent statistical significance. Values were expressed as mean-standard error of the mean and as percentage of the relaxation.

(f) Results

1. The relaxation effect of icariin on corpus cavernosum and abdominal aorta and EC₅₀

Icariin induce no contraction or relaxation in the basal equilibrium state of corpus cavrnosum and artery strips, but showed concentration-related relaxation response in corpus cavernosum and artery strips pe-contracted by PHE(corpus cavernosum strips: 10⁻⁶M; 1.23±0.82, 10⁻⁸M; 4.42±1.08, 10⁻⁴M; 23.12±1.36%) and (artery strips: 10⁻⁶M; 0.13±0.07, 10⁻⁵ M, 1.12±0.86, 10⁻⁴ M; 6.78±1.24%), respectively (p<0.001).(Table 1.) Solution above 10⁻³M of icariin could not be tested because of insolvency. Using logistic regression analysis the EC₅₀ relaxation effect on of corpus cavernosum and abdominal aorta were 4.67×10⁻⁴M, 2.7×10⁻³M receptively. The EC₅₀ of icariin on corpus cavernosum was 5.5 times lower than abdominal aorta.

TABLE 1
Relaxation effects of icariin on corpus cavernosum and arteries
muscle strips precontracted by PHE (5 × 10⁻⁶M)(%).

| Icariin | | | | | |
|---|---|---|---|---|---|
| conc. | | | | | P |
| (M) | 10⁻⁷ | 10⁻⁶ | 10⁻⁵ | 10⁻⁴ | Value |
| Corpus | 0.08 ± 0.04 | 1.23 ± 0.82 | 4.42 ± 1.08 | 23.12 ± | <0.001 |
| caver- | | | | 1.36 | |
| nosum | | | | | |
| (n = 8) | | | | | |
| Arteries | 0.02 ± 0.03 | 0.13 ± 0.07 | 1.12 ± 0.86 | 6.78 ± | <0.001 |
| (n = 8) | | | | 1.23 | |
| P value | >0.05 | 0.004 | <0.001 | <0.001 | |

Icariin rich fraction induce no contraction or relaxation in the basal equilibrium state of corpus cavrnosum and artery strips, but showed concentration-related relaxation response in corpus cavernosum and artery strips pre-contracted by PHE(corpus cavernosum strips: 0.1 mg/ml; 2.24±0.82, 0.5 mg/ml; 9.68±1.27, 1.0 mg/ml; 18.74±3.25%, 5.0 mg/ml; 44.24±4.35%, 10.0 mg/ml: 87.45±4.12%, 15.0 mg/ml: 94.23±4.36%) and (artery strips: 0.1 mg/ml; 0.12±0.08, 0.5 mg/ml: 1.34±0.98, 1.0 mg/ml: 4.12±3.35%, 5.0 mg/ml: 9.45±3.24%, 10.0 mg/ml: 23.05±4.21%, 15.0 mg/ml: 36.45=4.35%), respectively (p<0.001).(Table 1.).

TABLE 2
Relaxation effects of icariin rich fraction on corpus
cavernosum and arteries muscle strips precontracted by PHE
(5 × 10⁻⁶M - (%).

| Conc. | | | | | | | |
|---|---|---|---|---|---|---|---|
| (mg/ml) | 0.1 | 0.5 | 1.0 | 5.0 | 10.0 | 15.0 | P value |
| Corpus | 2.24 ± 0.82 | 9.86 ± 1.27 | 18.74 ± 3.25 | 44.24 ± 4.35 | 87.45 ± 4.12 | 94.23 ± 4.36 | <0.001 |
| cavernosum | | | | | | | |
| (n = 8 | | | | | | | |
| Arteries | 0.12 ± 0.08 | 1.34 ± 0.98 | 4.12 ± 3.35 | 9.45 ± 3.24 | 23.05 ± 4.21 | 36.45 ± 4.35 | <0.001 |
| (n = 8) | | | | | | | |
| P value | | <0.001 | <0.001 | <0.001 | <0.001 | <0.001 | |

Using logistic regression analysis the EC₅₀ relaxation effect on of corpus cavernosum and abdominal aorta were 4.08 mg/ml, 22.4 mg/ml repectively. The EC₅₀ of icariin rich fraction on corpus cavernosum strips was 5.8 times lower than abdominal aorta strips. Table 2.

2. Pharmaceutical mechanism of icariin on corpus cavernosum

(i) The relaxation effect of icariin on corpus cavernosum was inhibited (13.98%) by pretreatment with atropine(10⁻⁶) and by pretreatment with prostagladine inhibitor indomethacine(10⁻⁴M) (6.8%). However, the relaxation effect of icariin (10⁻⁴M) were not significantly inhibited by indomethacine and atropine(p>0.05). Table 3.

(ii) The relaxation effect of icariin on corpus cavernosum was significantly inhibited by de-endothelialization(32.18%), (p<0.01). The relaxation effects of icariin on corpus cavernosum were significantly inhibited by pretreatment with nitric oxide(NO) scavenge pyrogallol(10⁻⁴M) and NO synthesis inhibitor L-NNA(10⁻⁵ ) by 19.72, 25.95% respectively, (p<0.05). The relaxation effects of icariin on corpus cavernosum were significantly inhibited by pretreatment with methylene blue(10⁻⁴M) and ODQ(10⁻⁵M) by 41.82%, 50.48% respectively, (p<0.001). (Table 3.).

TABLE 3
Responses of deendothialization and pretreatment with L-NNA,
methylene blue, ODQ, pyrogallol, atropine and indomethacine on icariin
induced relaxation effects of corpus cavernosum muscle strips
precontracted by PHE (PHE; 5 × 10⁻⁶M).

| Conc. (M) | 10⁻⁶ | 10⁻⁵ | 10⁻⁴ | P value |
|---|---|---|---|---|
| Control | 1.23 ± 0.82 | 4.42 ± 1.08 | 23.12 ± 1.36 | |
| (n = 8) | | | | |
| De-endo | 0.82 ± 0.58 | 3.23 ± 0.89 | 15.68 ± 1.15 | <0.001 |
| (n = 5) | | | | |
| L-NNA | 0.85 ± 0.45 | 3.45 ± 0.73 | 18.12 ± 1.08 | <0.01 |
| (n = 5) | | | | |
| Methylene | 0.52 ± 0.68 | 2.02 ± 0.45 | 13.45 ± 1.12 | <0.001 |
| blue (n = 5) | | | | |
| ODQ | 0.42 ± 0.48 | 1.85 ± 0.35 | 10.45 ± 1.08 | <0.001 |
| (n = 5) | | | | |
| Atropine | 1.21 ± 0.58 | 4.11 ± 0.98 | 21.78 ± 1.06 | >0.05 |
| (n = 5) | | | | |
| Indomethacine | 1.18 ± 0.48 | 4.09 ± 1.08 | 20.12 ± 1.14 | >0.05 |
| (n = 5) | | | | |

(iii) ODQ(10⁻⁷M-10⁻⁴M) inhibitied relaxation effect of icariin (10⁻⁴ M) in dose dependent manner 4.33±1.22, 14.55±2.45, 48.02±3.78, 87.76±4.78%.(p<0.001),.

TABLE 4
Responses of pretreatment with various concentration of ODQ on
relaxation effects of icariin on corpus cavernosum muscle strips
precontracted by PHE (PHE; 5 × 10⁻⁶M).

| ODQ | | | | | |
|---|---|---|---|---|---|
| conc. | 10⁻⁷ | 10⁻⁶ | 10⁻⁵ | 10⁻⁴ | P value |
| Relaxation | 4.33 ± | 14.55 ± | 48.02 ± | 87.76 ± | <0.001 |
| effects of | 1.22 | 2.45 | 3.78 | 4.78 | |
| icariin (%) | | | | | |

With these results it is conclude that icariin has the relaxation effects on the corpus cavernosum and artery smooth muscle strips, and the potency of the relaxation effect of icariin on corpus cavernosum was significantly high than that of on artery smooth muscle strips. The pharmaceutical mechanism of icariin on corpus cavernosum smooth muscle mediated by synthesis and/or activation of NO-cGMP pathway.

Experiment 3: Studies of the inhibiting effect of icariin on PDE V enzyme

It is considered that the relaxation effect of icariin on corpus cavernosum is related to synthesis and/or activation of NO-cGMP pathway. NO or NO containing substance are diffused and penetrated into the cell of smooth muscle, a soluble guanylate cyclase of smooth muscle is thus activated and production of cGMP. cGMP is increased to induce relaxation of corpus cavernosum. cGMP is also decomposed into GMP by phosphodiesterase(PDE) to lose its activity. Accordingly, it is considered that PDE inhibitor increases cGMP concentration of smooth muscle and thus enhances relaxation action on smooth muscle. Since a large amount of PDE V type enzyme are distributed in the corpus cavernosum and platelets, the response on test drug was conventionally measured by isolating PDE V type enzyme from platelets.

(i) Isolation of PDE V isoenztme from human blood platelet

Platelet-rich plasma (PRP) was obtained by centrifuging 360 g of fresh human blood for 10 minutes. The PRP was then centrifuged at 1400 g for 10 minutes to sediment the platelets. The Platelets were re-suspended in 1 ml ice-cold homogenization buffer(HEPES 0.20 nM, sucrose 1 mM, EDTA 1 mM, phenymethyl sulfonylfluoride[PMSF] 1 mM, pH 7.2). The cell wall was disrupted by sonication, stood in cell lusate for 10 minutes and continuously centrifuged for 60 minutes. The supernatants were recovered and using PHARMACIA FPLC (Pharmacia Ltd. Milton Keynes isolated the enzyme. UK). The isolated enzyme was purified by Mono-Q anion exchange column(Pharmacia Ltd. Milton Keynes. UK). PDE V activity of the resulting enzyme was measured. The enzyme having activity was selected and used after storage in liquid nitrogen.

(iii) Determination of PDE enzyme inhibitor

All procedures for determining an activity of PDE enzyme inhibitor were made in ice. Assay buffer(Teis 40 mM, MgCl 10 mM, BSA 0.125 mg/ml, 2-mercaptoethanoal 3.75 mM, pH 8.0) was always newly prepared and used. The PDE V activity was determined using a modification of the two-step radioisotopes method with [³H]-cGMP. The inhibitory potency of compound against PDE V enzyme was determined by incubating the enzyme in presence of icariin in dimethyl sulfate(DMSO) with [³H]-cGMP as a substrate. The substrate, [³H]-cGMP(ca.28 ci/mmol; ca. 100,000 com/100 μl), prepared in assay buffer was used. 100 ul of cGMP was added in test tube to which 10 ul of drug(icariin 10⁻¹⁰M-10⁻⁴M) was added. The purified PDE V enzyme and assay buffer were added thereto and subjected to reaction at 30° C. for 20 minutes. Reactions were initiated by the addition of the radiolabeled substrate and Immersing the sample tubes in boiling water for 40-60 seconds stopped the enzyme reaction. The changed [³H]-cGMP was eluted with anion exchange column chromatography using 0.1N NaOH in AG1-X2 resin(Bio-Red). The radioactivity of the sample thus obtained was determined. The enzyme activity was determined in this manner.

(iv) Results

IC₅₀ values for inhibition of PDE V was determined from sigmoid curves, fitted to plots of enzyme activity versus log component concentration using a curve fitting program showed icariin significantly inhibited PDE V activity in a dose dependent manner (p<0 001), and IC₅₀ was 0.43 μM.

TABLE 5
Inhibiting activity of icariin on PDE V (%)

| Conc | 10⁻⁹M | 10⁻⁸M | 10⁻⁷M | 10⁻⁶M | 10⁻⁵M | 10⁻⁴M |
|---|---|---|---|---|---|---|
| Inhibiting | 0 | 0.2 | 42 | 63.2 | 87.9 | 99.7 |
| activity | | | | | | |

Conclusion

From the above results, it is concluded that icariin has a stronger relaxation effects on corpus cavernosal smooth muscle than arteries blood vessel. This proves that the relaxation effect is related to endothelial cell of corpus cavernosum, and production and activation of NO-cGMP pathway by the inhibition of PDE V enzyme and thus increases penile erection.

Experiment 4: Clinical test

This clinical test was conducted for 56 patients suffer from sexual dysfunction and who agreed to this clinical study of icariin using an international index of erectile function(IIEF-5) which was used for evaluating sexual function. The mean age of the patients was 36±6.7 years. The subjects included 7 diabetic patients(25%), 6 hypertension patients(21.4%), and 5 cardiac patient(17.9%). The packing of test drug and control drug was same manner. The patients had no information on the drugs used. The test drug(icariin 50 mg/tablet, 2 tablets/dose, 3 times/day) and control drug were administered to patients for one month randomly Prior to administering drugs, sexual function of all patients were evaluated by using IIEF. After administering drugs, sexual function changes of the patients were re-evaluated by using the IIEF. The comparison and analysis of the results were carried out.

Before administration, it was shown that confidence on penile erection and maintenance of erection(No 1) was 1.87±0.54, erection ability to interpose penis into vagina(No 2) was 1.89±0.67, the ability to maintain erection after vaginal penetration during their sexual intercourse(No 3) was 1.82=0.65, and the inability to maintain erection and maitainance of erection during their sexual intercourse(No 4) was 1.89±0.61, and satisfaction with their sexual lives(No 5) was 1.79±0.62, respectively.

After administration, it is shown that confidence on penile erection and maintenance of erection(No 1) was 3.32±0.39 for icariin tablet, 2.04±0.63 for control drug. The erection ability to interpose penis is penitrated into vaginal during intercourse(No 2) 3.11±0.32 for icariin tablet, and 2.05±0.88 for control drug, the ability to maintain erection after vaginal penetration during their sexual intercourse(No 3) was 3.18±0.47 for icariin tablet, and 2.17±0.97 for control drug, and inability to maintain erection and maintains erection during sexual intercourse(No 4) was 3.25±0.47 for icariin tablet, and 2.18±0.92 for control drug, and satisfaction with sexual lives(No 5) was 3.36±0.47 for icariin tablet, and 2.14±0.95 for control drug. Those results prove that the clinical effects were statistically significant(p<0.01).

TABLE 6
Effects of icariin tablet on patients with sexual dysfunction evaluated by IIEF

| | No 1 | | No 2 | | No 3 | | No 4 | | No 5 | |
|---|---|---|---|---|---|---|---|---|---|---|
| | Pre | Post | Pre | Post | Pre | Post | Pre | Post | Pre | Post |
| IIEF | Tx | Tx | Tx | Tx | Tx | Tx | Tx | Tx | Tx | Tx |
| Icariin | 1.82 ± | 3.32 ± | 1.89 ± | 3.11 ± | 1.82 ± | 3.18 ± | 1.89 ± | 3.25 ± | 1.79 ± | 3.36 ± |
| tablet | 0.54 | 0.39 | 0.67 | 0.32 | 0.65 | 0.47 | 0.61 | 0.47 | 0.62 | 0.47 |
| (n = 26) | | | | | | | | | | |
| Control | 1.82 ± | 2.04 ± | 1.89 ± | 2.07 ± | 1.82 ± | 2.17 ± | 1.89 ± | 2.18 ± | 1.79 ± | 2.14 ± |
| (n = 26) | 0.54 | 0.63 | 0.67 | 0.88 | 0.65 | 0.97 | 0.61 | 0.92 | 0.62 | 0.95 |
| P value | | 0.002 | | 0.001 | | 0.01 | | 0.002 | | 0.001 |

The overal clinical effectiveness was 75.4% for icariin tablet and 35.4% for control drug. There were no significant side effects except that 4 patient show a mild gastric reaction. There were not observed significant differences in clinical effects and side effects from diabetic, hypertension and cardiac patients.

It is apparent from the results of the above teat that icariin has the relaxation effects on corpus cavernosum smooth muscle and arteries. NO-cGMP pathway as well as inhibition of PDE V enzyme to increase the concentration of cGMP mediates the mechanism of icariin on corpus cavernosum smooth muscle. It is implied that icariin may enhance penile erection during sexual arousal in man with erectile dysfunction.

As set above, icariin has a relaxation effect on corpus cavernosal smooth muscle to improve a sexual function as well as a relaxation effect on vascular smooth muscle and artery blood vessel. Further, it has no since effect in the treatment of sexual dysfunction and can be effectively and safely applied to patients suffering from diabetic, hypertension and cardiac problem. Therefore, the pharmaceutical composition of the present invention comprising icariin can be effectively and safely used as a medical agent for preventing and treating a sexual dysfunction and vasculargenic disease.

What is claimed is:

1. A pharmaceutical composition for preventing and treating a sexual dysfunction and vasculargenic disease comprising a therapeutically effective amount of icariin as an active component.

2. A use of icarrin for the manufacture of the pharmaceutical for preventing and treating a sexual dysfunction and vasculargenic disease.

3. A pharmceaufical composition according to claim 1 or a use according to claim 2, wherein said pharmaceutical composition or the pharmaceutical contains 1-500 mg icariin.

4. The pharmaceutical composition or use of any one according to claim 1-3, wherein the composition or pharmaceutical includes tablet, pill, suspension, solution, patch, capsular, dispersion, ointment, cream, chewing gum, spary, injection and granular.

5. The pharmaceutical composition or use of claim 1-3, wherein the composition or pharmaceutical is administered in the form of oral, muscle and hypodermic injection, skin mucosa, rectum, urethra, vaginal and vein.

6. A method of the prevention or treatment of a sexual dysfunction and a vasculargenic disease which comprising administrating a prevention or treatment amount of icariin or a pharmaceutical composition containing icariin to a patient who needs.
